SEQUENCE LISTING (1 sequence)

SEQ ID NO: 1 - protein, 326 aa, Homo sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

(19) United States
(12) Patent Application Publication
(10) Pub. No.: US 2006/0040249 A1
(43) Pub. Date: Feb. 23, 2006

(54) Screening method involving transfected primary cells

(76) Inventors: Manfred Auer, Moedling (AT); Thomas Jung, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/526,284

(22) PCT Filed: Sep. 25, 2003
(86) PCT No.: PCT/EP03/10681

§ 371 Date: Mar. 1, 2005

Related U.S. Application Data
(60) Provisional application No. 60/413,705, filed on Sep. 26, 2002.

(51) Int. Cl. C12Q 1/00; C12Q 1/68; C12N 5/08

(52) U.S. Cl. 435/4; 435/6; 435/372

(57) ABSTRACT

A screening assay for identifying an agent that modulates T cells.

The present invention relates to a screening assay, e.g. to an assay for identifying an agent that modulates T cells.

Cellular screening and biological profiling of compounds ideally require intact primary cells rather than tumour cell lines. Tumour cell lines may lose their physiological susceptibilities towards extracellular as well as intracellular stimuli and show an autonomous, unspecific gene activation. In addition, many of the common tumour cell lines are of non-human origin. In comparison, primary cells are completely intact, resting and are fully responsive to physiological stimuli.

We have now found that primary human T cells transfected with biological active molecules, e.g. nucleic acids or proteins, are useful tools for screening and/or profiling compounds interfering with these biological active molecules. By e.g. co-transfecting either c-Rel or p65, two members of the NF-κB family, together with e.g. a reporter gene, it was shown that the reporter gene is activated by each of the transfected NF-κB molecules. In addition, we have found that both, c-Rel and p65, are functionally active in primary human T cells and induce a Th1 cytokine response and enhanced proliferation to mitogenic stimuli.

The present invention provides useful tools for, e.g. cellular, screening assays, biological profiling of drug compounds and high-throuphput screening assays (HTS assays).

In one aspect the present invention provides a cellular assay for identifying an agent that modulates T cells comprising the steps of:
a) providing primary T cells transfected with a biological active molecule selected from the group consisting of nucleic acid, protein, peptide, polysaccharide and lipid,
b) stimulating the transfected T cells of step a) in the absence and in the presence of a candidate compound for a sufficient period of time,
c) detecting the amount of a cytokine produced by the T cells and/or the proliferation of the T cells and/or the amount of a reporter molecule,
d) determining whether there is a difference in the amount of cytokine produced and/or in the amount of proliferation and/or in the amount of reporter molecule in the absence and in the presence of a candidate compound, and
e) choosing an agent from said candidate compound as determined in step d).

The assay of the present invention has the advantage that primary T cells, e.g. primary human T cells, may be used, which are characterized e.g. in that
they remain intact cells,
they remain resting after transfectiion, e.g. in a non-proliferating stage, and
they are fully responsive to physiologic stimuli.

T cells used in the present invention preferably are human T cells, e.g. freshly isolated T cells or frozen T cells which are thawed. T cells are purified from e.g. natural sources by methods as conventional, e.g. PBMC (peripheral blood monocyte cells) purified by methods as conventional, e.g. by rosette formation with sheep red blood cells.

A biological active molecule according to the present invention may be any (bio)chemical molecule, e.g. a molecule with a certain role in a physiologic pathway or context and includes a biological active molecule selected from the group consisting of nucleic acid, protein, peptide, polysaccharide and lipid.

The biological active molecule preferably is a DNA, e.g. DNA encoding a molecule of the NF-κB pathway, such as DNA encoding for c-Rel or p65, e.g. human c-Rel or human p65. The DNA codes for the full length of a biological active molecule, e.g. protein, or for fragments or equivalents thereof. A fragment is to be understood as a part of the full length molecule but with retained biological activity. An equivalent is a sequence with homology to the biological active molecule, which homology is such, that biological activity of said molecule is retained.

In a preferred aspect the biological active molecule is a DNA, preferably a DNA encoding a molecule of the NF-κB pathway.

In another preferred aspect the biological active molecule is a protein, preferably a HuR protein.

In case the biological active molecule is a DNA, it may be present e.g. as an expression construct wherein the DNA encoding the biological active molecule is inserted into a plasmid, optionally together with an appropriate inducible specific promoter and/or a reporter gene. A reporter gene may also be provided alone by insertion of DNA encoding a reporter molecule (=reporter gene) into a plasmid (=reporter gene construct). A reporter gene includes e.g. a luciferase gene, alkaline phosphatase or a fluorescence protein, e.g. GFP (=green fluorescence protein). The amount of reporter gene, e.g. the amount of expressed reporter gene, is detectable by a method as conventional. The expression construct may be co-transfected with the reporter construct into the T cells or can be operatively linked to a reporter gene in the same construct, e.g. plasmid.

In case the biological active molecule is a protein or peptide, this protein or peptide may be transfected into the T cells as such or in pre-labeled form, e.g. bearing a fluorescence label. The protein or peptide may be isolated from natural sources or may be generated by a chemical or recombinant method.

The biological active molecule may be transfected into the T cells by use of a transfection method which is efficient for primary T cells, preferably a non-viral transfection method. The preferred transfection method used according to the present invention retains the characteristic of primary T cells, e.g. the transfected T cells remain intact, are resting and fully responsive to physiologic stimuli.

In a preferred aspect the transfection of T cells is accomplished by use of an electrical current, e.g. an electrical current of 1.0 to 2.5 Ampere and an electrical field strength between 2 to 10 kV/cm for 5 to 200 μs.

The transfected primary T cells are stimulated so that the amount of a cytokine produced, e.g. interleukin-2 or IFN-γ, and/or the amount of proliferation and/or the amount of reporter molecule may be detected in the absence and in the presence of a candidate compound.

Further parameters like e.g. time for stimulation, number of cells required, solvent used etc. may be optimized according, e.g. analogously, to a method as conventional.

The time for stimulation is e.g. from 2 to 48 hours, preferably about 18 hours.

The ratio of DNA to T cells is e.g. 2 μg of DNA-plasmid for 5×10⁶ T cells.

The solvent used for transfection is e.g. a solvent as provided by the manufacturer of the Nucleofector™ kit or is PBS.

Appropriate methods for the detection of e.g. a reporter molecule include fluorescence spectroscopy with a particular focus on applications with single molecule sensitivity e.g. Fluorescence Correlation Spectroscopy (FCS), Fluorescence Intensity Distribution Analysis (FIDA) or applications based on the determination of Fluorescence Anisotropy or Fluorescence Resonance Energy Transfer (FRET), e.g. as described in Kask P. et al, Biophys. J. (2000) 78 (4), 1703-1713. Proliferation can be determined according to a method as conventional, such as e.g. the beta counter measurement of ³H-tymidine incorporation after incubation of the cells with ³H-tymidine. Cytokines may be detected according to methods as conventional, such as using a detection molecule. Appropriate detection molecules are known or may be found according, e.g. analogously, to a method as convential and include e.g. horseradish peroxidase substrates, alkaline phosphatase substrates, luciferase substrates, time resolve fluorescence substrates, e.g. using lanthanide-labels, and enhancement solutions, and polymerase chain reaction solutions., e.g. in case of an enzyme as a detection molecule, measuring the enzymatic activity, or, in case of a labeled reagent, measuring a label-specific effect, e.g. in case of fluorescence labeling, measuring the label-specific effect by appropriate luminescence/fluorescence determination methods at appropriate wavelengths, e.g. including methods as conventional.

A candidate compound is a compound which may modulate T cells, especially human T cells, and includes compound(s)(libraries) from which its influence on the T cells can be determined. Compound (libraries) include for example oligopeptides, polypeptides, proteins, antibodies, mimetics, small molecules, e.g. low molecular weight compounds (LMW's).

An agent is a candidate compound which influences (inhibits) the amount of a cytokine produced by the T cells and/or the proliferation of the T cells and/or the amount (expression) of the reporter gene and/or its products detected/determined in step d). An agent includes oligopeptides, polypeptides, proteins, antibodies, mimetics, small molecules, e.g. low molecular weight compounds (LMW's).

In a further aspect the present invention provides a kit for identifying an agent that modulates T cell comprising as components:
a) primary T cells transfected with a biological active molecule selected from the group consisting of nucleic acid, protein, peptide, polysaccharide and lipid,
b) stimulation means, and
c) detection means for cytokines.

Stimulation means include those as conventional, e.g. monoclonal antibodies, such as anti-CD3 and/or anti-CD28 monoclonal antibodies.

Detection means Include those as conventional, e.g. as indicated herein.

In a further aspect the present invention provides the use of primary T cells in a cellular screening assay.

In another aspect the present invention provides the use of primary T cells for biological profiling of compounds.

In another aspect the present invention provides the use of primary T cells in a high throughput screening assay.

In another aspect the present invention provides the use of the Nucleofector™ technology in a method of the present invention.

An agent of the present invention may exhibit pharmacological activity and is therefore useful as a pharmaceutical against e.g components of the NF-κB signaling pathway.

An agent of the present invention may show therapeutic activity against e.g components of the NF-κB signaling pathway.

For pharmaceutical use an agent of the present invention for treatment includes one or more, preferably one, agent of the present invention, e.g. a combination of two or more agents of the present invention.

In another aspect the present invention provides an agent for use as a pharmaceutical.

In a further aspect the present invention provides a pharmaceutical composition comprising an agent identified by a method according to the present invention as an active ingredient in association with at least one pharmaceutical excipient.

In another aspect the present invention provides the use of an agent of the present invention for the manufacture of a medicament, e.g. a pharmaceutical composition, for the treatment of a disorder having an etiology associated with the production of a substance, e.g. an inflammatory acting (causing/enhancing) substance, selected from the group consisting of cytokine, growth factor, proto-oncogene or viral protein

The pharmaceutical compositions according to the present invention may be used for the treatment of disorders as indicated herein. Preferably said substance is selected from the group consisting of IL-1, IL-2, IL-3, IL-4, IL-8, GM-CSF, TNF-α, VEGF, AT-R1, Cox-2, c-fos and c-myc. Treatment includes treatment and prophylaxis.

For such treatment, the appropriate dosage will, of course, vary depending upon, for example, the chemical nature and the pharmacokinetic data of an agent of the present invention employed, the individual host, the mode of administration and the nature and severity of the conditions being treated. However, in general, for satisfactory results in larger mammals, for example humans, an indicated daily dosage is in the range from about 0.01 g to about 1.0 g, of an agent of the present invention; conveniently administered, for example, in divided doses up to four times a day.

An agent of the present invention may be administered by any conventional route, for example enterally, e.g. including nasal, buccal, rectal, oral administration; parenterally, e.g. including intravenous, intramuscular, subcutanous administration; or topically; e.g. including epicutaneous, intranasal, intratracheal administration; e.g. in form of coated or uncoated tablets, capsules, injectable solutions or suspensions, e.g. in the form of ampoules, vials, in the form of creams, gels, pastes, inhaler powder, foams, tinctures, lip sticks, drops, sprays, or in the form of suppositories.

An agent of the present invention may be administered in the form of a pharmaceutically acceptable salt, e.g. an acid addition salt or metal salt; or in free form; optionally in the form of a solvate. An agent of the present invention in the form of a salt may exhibit the same order of activity as an agent of the present invention in free form; optionally in the form of a solvate.

An agent of the present invention may be used for pharmaceutical treatment according to the present invention alone, or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

In another aspect the present invention provides a pharmaceutical composition comprising an agent of the present Invention in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a pharmaceutical composition according to the present invention, further comprising another pharmaceutically active agent.

Such compositions may be manufactured according, e.g. analogously, to a method as conventional, e.g. by mixing, granulating, coating, dissolving or lyophilizing processes. Unit dosage forms may contain, for example, from about 0.5 mg to about 2000 mg, such as 1 mg to about 500 mg, e.g. 0.00625 mg/kg to about 12.5 mg/kg.

In a further aspect the present invention provides an assay for identifying an agent that modulates T cells comprising the steps of:
a) providing primary T cells,
b) providing a biological active molecule selected from the group consisting of nucleic acid, protein, peptide, polysaccharide and lipid, and optionally a reporter molecule,
c) transfecting the primary T cells of step a) with a biological active molecule of step b), and optionally with a reporter molecule, in using a transfection method which is efficient for primary T cells
d) stimulating the transfected T cells in the absence and in the presence of a candidate compound which might modulate the T cells for a sufficient period of time,
e) detecting the amount of a cytokine produced by the T cell and/or the proliferation and/or the co-transfected reporter molecule,
f) determining whether there is a difference in the cytokine produced and/or in proliferation and/or in the amount of reporter molecule in case a candidate compound was present or absent, and
g) choosing an agent from said candidate compound as determined in step f), e.g. for use as a pharmaceutical.

In the following examples all temperatures are in degree Centigrade (° C.) and are uncorrected.

The following ABBREVIATIONS are used:

| | |
|---|---|
| BSA | bovine serum albumin |
| CBD | chitin binding domain |
| CLSM | confocal laser scanning microscopy |
| DAPI | 4,6-diamino-2-phenylindole |
| Cy5 | commercial available dye |
| ELISA | enzyme linked immunosorbent assay |
| FACS | fluorescence activated cell sorting |
| FIDA | fluorescence intensity distribution analysis |
| HuR | Hu-antigen R |
| IL-2 | Interleukin 2 |
| mab(s) | monoclonal antibody(ies) |
| MESNA | 2-mercaptoethanesulfonic acid |
| NF-κB | nuclear factor of kappa light chain |
| | enhancer in B cells |
| PMA | phorbol myristate acetate |
| RT | room temperature |
| TCR | T cell receptor |

### DESCRIPTION OF THE FIGURES

FIG. 1: Transfection of DNA plasmids into resting human T cells Overexpression of c-Rel and p65 enhances proliferation and production of IL-2 and IFNγ. Resting T cells are transfected with full length CD25 to monitor transfection efficacy. Dotted line shows CD25 expression in non-ransfected cells, bold line shows expression of CD25 24 hours after transfection.

FIG. 2: Transfection of DNA plasmids into resting human T cells Resting T cells are transfected with a NF-κB-luciferase reporter gene construct. In addition, full length p65 and c-Rel are co-transfected as a control plasmid. After 4 hours cells are activated either with anti-CD3 and anti-CD28 mabs (=TCR) or with PMA and ionomycin (=PI) or kept in medium, unstimulated, for 18 hours. Luciferase activity is measured by a luminometer. Results show mean ±SD of duplicates of one experiment out of three.

FIG. 3: Transfection of DNA plasmids into resting human T cells Resting T cells are transfected with a control plasmid, full length p65 or c-Rel. 24 hours later, the cells are activated with anti-CD3 and anti-CD28 mabs and proliferation is measured after 3 days by ³H-thymidine incorporation (mean±SD of triplicates). On day 2, supernatants are harvested and analyzed for IL-2 and IFNγ production by ELISA. Data show mean±SD of duplicates of one experiment out of three.

FIG. 4: Transfection of proteins into resting human T cells Freshly isolated T cells are incubated with Cy5-labeled HuR (final concentration 200 nM) and are either pulsed (bottom pictures) or left non-transfected (upper pictures). Images show example cells after counterstaining with DAPI.

FIG. 5: Transfection of proteins into resting human T cells Cy-5 labeled HuR and unlabeled HuR are transfected in freshly isolated T cells, IL-2 production (FIG. 5A) and proliferation (FIG. 5B) are determined as described in example 2.

### EXAMPLES

### Example 1

### Transfection of Primary T Cells with DNA Plasmids

a) Construction of Plasmids for NF-κB Assays

Full length human c-Rel (Genbank Acc No X75042), p65 (Acc No M62399) and CD25 (Acc No NM_{—}000417) are amplified from a cDNA library of activated T cells by PCR and expression constructs created by ligation into pcDNA3.1 TOPO (Invitrogen, Carlsbad, Calif.) that are verified by DNA sequencing. A reporter gene construct containing 5×NF-κB binding sites upstream (5×NF-κB/Luc) of the luciferase gene is purchased from Stratagene (La Jolla, Calif., No 219077).

b) Transfection of T Cells

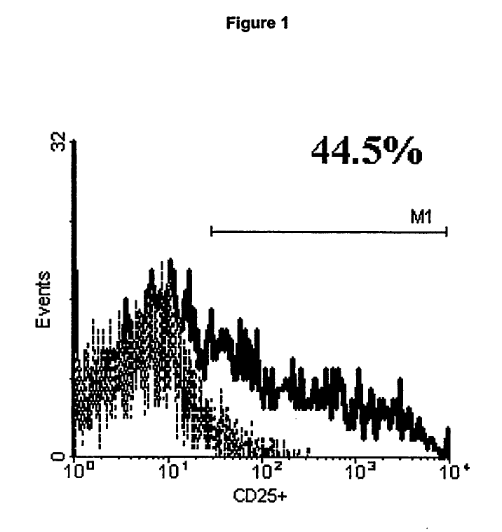

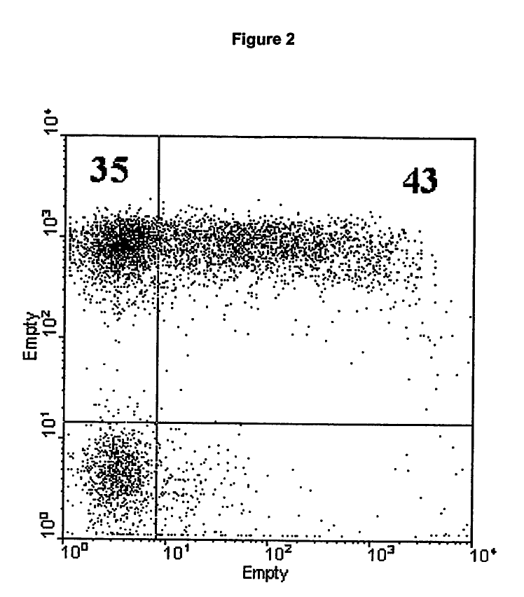

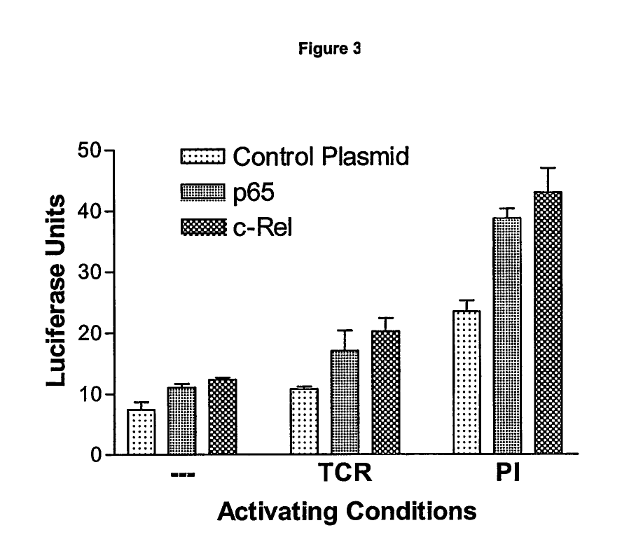

Purified T cells are electroporated using the Nucleofector™ Technology (Amaxa, Cologne, Germany). Briefly, 5×10⁶ T cells in 100 μl transfection solution as provided by the Nucleofector™ kit are co-transfected with 2 μg of the NF-κB cis reporter construct and 1 μg of the c-Rel, p65 or control (pcDNA3.1 empty vector) expression construct. 4 hours after transfection, cells are activated for 18 hours and luciferase activity is detected by a Wallac luminometer. In parallel, cells transfected only with the expression constructs are activated after transfection with anti-CD3 and anti-CD28 mabs to induce proliferation and cytokine production. Transfection efficacies are routinely controlled by FACS analysis of cells transfected with the CD25 expression construct 24 hours after transfection. Proliferation is measured after incubation of 0.5 μCi ³H-thymidine for 16 hours in a beta counter (see e.g. FIGS. 1, 2 and 3). The cells are efficiently transfected with the DNA plasmid encoding reporter gene and NF-κB transcription factors c-Rel or p65.

### Example 2

### Transfection of Primary T Cells with Proteins

a) Expression, Purification and Labeling of HuR

The preparation of recombinant full length human HuR encompassing amino acids 1-326: MSNGYEDHMAEDCRGDIGRTNLIVNYLPQNMTQDELRSLFSSIGEVESAKLIRDKVAGHSLG YGFVNYVTAKDAERAINTLNGLRLQSKTIKVSYARPSSEVIKDANLYISGLPRTMTQKDVEDM FSRFGRIINSRVLVDQTTGLSRGVAFIRFDKRSEAEEAITSFNGHKPPGSSEPITVKFMNPN Q189NKNVALLSQLYHSPARRFGGPVHHQAQRFRFSPMGVDHMSGLSGVNVPGNASSGW CIFIYNLGQDADEGILWQMFGPFGAVTNVKVIRDFNTNKCKGFGFVTMTNYEEMM AIASLNGYRLGDKILQVSFKTNKSHK³²⁶ using the IMPAC™—CN System (New England Biolabs) is performed as described previously.

Briefly, full-length human HuR is PCR-amplified from cDNA prepared from, both, activated human T-lymphocytes as well as human monocyte-derived dendritic cells, and cloned directionally into the Ndel and Sapl sites of the vectors pTXB1 and pTYB1, allowing C-terminal fusion with the Intein-CBD tag without insertion of any additional amino acid. These constructs are transformed into the host strain E.coli ER2566 (New England Biolabs) for plasmid DNA amplification and protein expression. The recombinant HuR-lntein-CBD fusion protein is purified from bacterial cell lysates by affinity chromatography with chitin agarose. After extensive washing steps with lysis buffer at high salt concentrations and in the presence of 0.1% Triton-X-100 to reduce unspecific binding, the recombinant target protein is eluted by induction of on-column cleavage with MESNA (50 mM) and incubation at 4°. The concentration of the purified protein is determined based on a commercially available protein assay according to method of Bradford et al. The purity of the protein is monitored by denaturing, non-reducing SDS-PAGE, followed by silver staining as well as by analytical RP-HPLC.

A shorter variant of human HuR (amino acid 1 to 189 encompassing the first two RNA recognition motifs (RRM)) is prepared using the IMPACT-TWIN System by directional cloning into the restriction sites Ndel and Sapl in analogy to the preparation of full length human HuR as described. According to the literature and data from related proteins, this protein should be equally active in RNA-binding as compared to the full-length protein, but be compromised in its transport between nucleus and cytosol, as reviewed by e.g. Brennan and Steitz (2001). This may be verified in an 2D-FIDA-r assay.

Subsequently, full length HuR is 1:1 fluorescently labeled at its C-terminus via e.g. the C-terminal MESNA thioester.

b) Transfection of T Cells

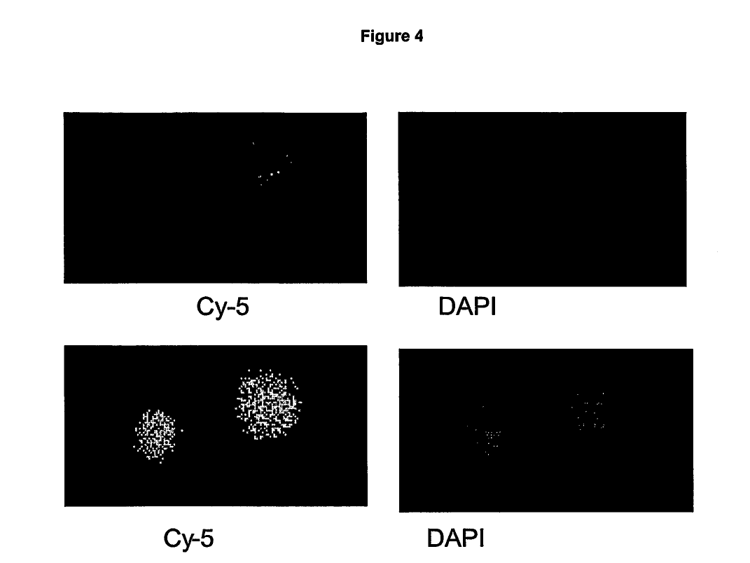

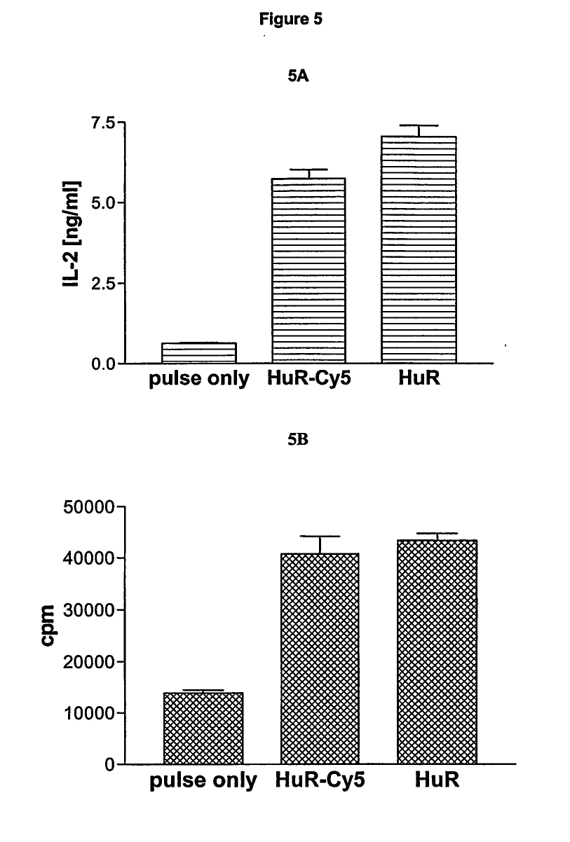

Resting T cells are transfected using the Nucleofector™ technology (Amaxa, Cologne, Germany). Briefly, 1×10⁶ Tcells are mixed with Cy5-labeled recombinant HuR (200 nM final concentration) in 100 μl PBS. Non-labeled HuR or BSA is used as a control. The cells are exposed to pulse U14 according to the manufacturer's instructions. After transfection, 1 ml of warm culture medium is added and cells are cultured in 2 ml of complete culture medium for 45 minutes. Cells are washed, fixed in 1% paraformaldehyde and cytospins are performed. Cells are air-dried for 30 minutes and embedded in mounting medium (Vectashield®). Examination is carried out using a LSM5 Pascal confocal laser scanning microscope (Zeiss, Axiovert 200M). For observation, a Plan-Apochromat 63x/1.4 oil lense is used. Signals are recorded using the following wavelengths: excitation 633 nm and emission 667 nm for Cy5; excitation 358 nm and emission 461 nm for DAPI. A series of CLSM images are taken at five random locations on control and treatment slide pieces. Transfected T cells are activated with soluble anti-CD3 plus anti-CD28 mabs to proliferate and to produce IL-2. Supernatants are harvested after 48 hours of culture and analyzed using a human IL-2 specific ELISA and proliferation is determined after 72 hours by ³H-thymidine incorporation (see e.g. FIGS. 4, 5A and 5B). The cells are efficiently transfected with HuR protein.

1. A cellular assay for identifying an agent that modulates T cells comprising the steps of:
a) providing primary T cells transfected with a biological active molecule selected from the group consisting of nucleic acid, protein, peptide, polysaccharide and lipid,
b) stimulating the transfected T cells of step a) in the absence and in the presence of a candidate compound for a sufficient period of time,
c) detecting the amount of a cytokine produced by the T cells and/or the proliferation of the T cells and/or the amount of a reporter molecule,
d) determining whether there is a difference in the amount of cytokine produced and/or in the amount of proliferation and/or in the amount of reporter molecule in the absence and in the presence of a candidate compound, and
e) choosing an agent from said candidate compound as determined in step d).

2. A method according to claim 1 wherein the biological active molecule is a DNA.

3. A method according to claim 1 wherein the biological active molecule is a protein.

4. A method according to claim 1 wherein transfection in step a) is accomplished by use of a non-viral transfection method.

5. A kit for identifying an agent that modulates T cell comprising as components:
a) primary T cells transfected with a biological active molecule selected from the group consisting of nucleic acid, protein, peptide, polysaccharide and lipid,
b) stimulation means, and
c) detection means for cytokines.

6. The use of transfected primary T cells in a cellular screening assay.

7. The use of transfected primary T cells for biological profiling of compounds.

8. The use of transfected primary T cells in a high throughput screening assay.
